(19) United States
(12) Patent Application Publication
(10) Pub. No.: US 2014/0300717 A1
(43) Pub. Date: Oct. 9, 2014

(54) ENDOSCOPE APPARATUS

(71) Applicant: OLYMPUS CORPORATION, Tokyo (JP)

(72) Inventor: Tomohiro YOKOHAMA, Tokyo (JP)

(73) Assignee: Olympus Corporation, Tokyo (JP)

(21) Appl. No.: 13/858,327

(22) Filed: Apr. 8, 2013

(51) Int. Cl. A61B 1/045

(52) U.S. Cl. CPC A61B 1/045
USPC 348/68; 348/65

(57) ABSTRACT

An endoscope apparatus has an insertion portion having an image pickup device in a distal end portion, and a main body portion. The endoscope apparatus has a waveform shaping circuit that is provided in the distal end portion of the insertion portion, a stabilizing circuit that stabilizes a power supply voltage to the waveform shaping circuit, a load circuit that is connected to a power supply line that supplies the power supply voltage of the waveform shaping circuit, and a current drawing circuit that draws a predetermined current to the load circuit from the power supply line in response to an operation state of the waveform shaping circuit so that a potential difference between a power supply voltage of the waveform shaping circuit in a non-operation time period of the waveform shaping circuit and a power supply voltage in an operation time period of the waveform shaping circuit becomes small.

### BACKGROUND OF THE INVENTION

1. Field of the Invention

The present invention relates to an endoscope apparatus having an image pickup device at a distal end portion of an insertion portion.

2. Description of the Related Art

Conventionally, endoscopes apparatuses have been widely used in an industrial field and a medical field. An image pickup device is disposed at a distal end of an insertion portion, and an endoscopic image obtained by an image being picked up by the image pickup device is displayed on a display apparatus of a main body portion connected to the insertion portion.

In an endoscope, the pickup device such as a CCD is driven via a thin and long signal line inserted through the insertion portion, and therefore, several kinds of clock signals such as a horizontal transfer signal which is an especially high-speed signal is inputted into the image pickup device via a waveform shaping circuit mounted on an endoscope distal end portion.

Normally, for the clock signal which is inputted into a wave form shaping circuit, a clock output time period and a clock stop time period are provided. In the clock output time period, a rectangular wave signal with a high frequency is inputted into the waveform shaping circuit, whereas in the clock stop time period, a constant DC voltage is inputted into the waveform shaping circuit.

The image pickup device that is a load circuit connected to the waveform shaping circuit has a large capacitive component, and therefore, during the clock output time period, a large amount of current for charging the load capacity flows into the load circuit. During the clock stop time period, the load circuit is already charged, and therefore, a current does not flow. Namely, a difference occurs to the current consumption of the waveform shaping circuit during the clock output time period and during the clock stop time period.

A power supply of the waveform shaping circuit is supplied through a long power supply line from a constant voltage source provided in the main body portion of an endoscope. When the difference occurs to the current consumption of the waveform shaping circuit as described above, a difference occurs to the voltage drop amount of the power supply line for supplying power supply, and therefore, a potential difference occurs to the power supply voltage in a power supply input terminal of the waveform shaping circuit during the clock output time period and during the clock stop time period.

In order to decrease the potential difference, and make the power supply voltage of the waveform shaping circuit to operate the waveform shaping circuit stably, a stabilizing circuit for a power supply voltage is mounted on the distal end portion of the endoscope insertion portion, as disclosed in, for example, Japanese Patent Application Laid-Open Publication No. 2002-562.

### SUMMARY OF THE INVENTION

An endoscope apparatus of one aspect of the present invention is an endoscope apparatus having an insertion portion having an image pickup device in a distal end portion, and a main body portion, and has a waveform shaping circuit that is provided in the distal end portion of the insertion portion and shapes a waveform of a drive signal to the image pickup device, a stabilizing circuit that stabilizes a power supply voltage to the waveform shaping circuit, a load circuit that is connected to a power supply line that supplies the power supply voltage of the waveform shaping circuit, and a current drawing circuit that draws a predetermined current to the load circuit from the power supply line in a non-operation time period of the waveform shaping circuit, so that the power supply voltage of the waveform shaping circuit in the non-operation time period of the waveform shaping circuit becomes equal to a power supply voltage in an operation time period of the waveform shaping circuit.

An endoscope apparatus of one aspect of the present invention is an endoscope apparatus having an insertion portion having an image pickup device in a distal end portion, and a main body portion, and has a waveform shaping circuit that is provided in the distal end portion of the insertion portion and shapes a waveform of a drive signal to the image pickup device, a stabilizing circuit that stabilizes a power supply voltage to the waveform shaping circuit, and a variable power supply circuit that reduces the power supply voltage which is supplied to the waveform shaping circuit in a non-operation time period of the waveform shaping circuit, so that the power supply voltage of the waveform shaping circuit in the non-operation time period of the waveform shaping circuit becomes equal to the power supply voltage of the waveform shaping circuit in an operation time period of the waveform shaping circuit.

### BRIEF DESCRIPTION OF THE DRAWINGS

FIG. 1 is a schematic configuration diagram of an endoscope apparatus according to a first embodiment of the present invention;

FIG. 2 is a waveform chart of various signals in an endoscope apparatus 1, according to the first embodiment of the present invention;

FIG. 3 is a schematic configuration diagram of an endoscope apparatus according to modification 1 of the first embodiment of the present invention;

FIG. 4 is a schematic configuration diagram of an endoscope apparatus according to modification 2 of the first embodiment of the present invention;

FIG. 5 is a schematic configuration diagram of an endoscope apparatus according to modification 3 of the first embodiment of the present invention;

FIG. 6 is a schematic configuration diagram of an endoscope apparatus according to a second embodiment of the present invention;

FIG. 7 is a waveform chart of various signals in the endoscope apparatus, according to the second embodiment of the present invention;

FIG. 8 is a schematic configuration diagram of an endoscope apparatus according to modification 1 of the second embodiment of the present invention; and

FIG. 9 is a schematic configuration diagram of an endoscope apparatus according to modification 2 of the second embodiment of the present invention.

### DETAILED DESCRIPTION OF THE PREFERRED EMBODIMENTS

Hereinafter, embodiments of the present invention will be described with use of the drawings.

### First Embodiment

### Configuration

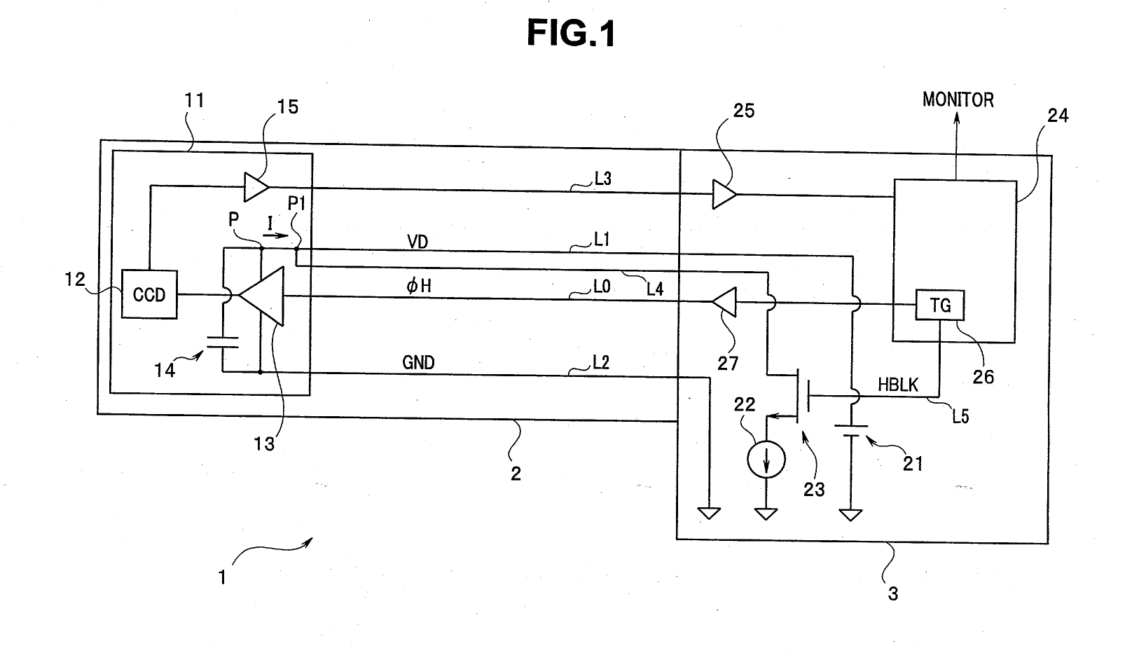

FIG. 1 is a schematic configuration diagram of an endoscope apparatus according to a first embodiment of the present invention. Note that FIG. 1 only shows a configuration relating to the present invention, and does not show components (for example, components such as a bending control mechanism and an operation section) that do not relate to the present invention.

An endoscope apparatus 1 is configured by having an insertion portion 2, and a main body portion 3 connected to a proximal end of the insertion portion 2. Though not illustrated here, a monitor (not illustrated) for displaying an endoscopic image is connected to the main body portion 3.

Note that the insertion portion 2 may be detachably connected to the main body portion 3. Furthermore, the monitor not illustrated may be provided at the main body portion 3 itself.

A distal end portion 11 of the insertion portion 2 is provided with a CCD image sensor (hereinafter, called a CCD) 12 as an image pickup device. An objective optical system (not illustrated) for forming an image of an object image on an image pickup surface of the CCD 12 is provided at the distal end portion 11. Namely, the endoscope apparatus 1 has the insertion portion 2 having the CCD 12 as the image pickup device at the distal end portion 11, and the main body portion 3.

Various pulse signals for drive from the main body portion 3 are inputted into the CCD 12 via a signal line. Various pulse signals include a reset gate signal, horizontal transfer signals φH1 and φH2, a vertical transfer signal and the like. Here, one horizontal transfer signal φH which especially and significantly influences variation of a power supply voltage of a waveform shaping circuit, has a high frequency and is inputted into the CCD 12 will be described.

The horizontal transfer signal φH of a signal line L0 is inputted into the CCD 12 via a waveform shaping circuit 13. The waveform shaping circuit 13 is a comparator that outputs a rectangular wave signal subjected to waveform shaping, by comparison of an inputted signal and a set threshold value. As shown in FIG. 1, the waveform shaping circuit 13 is a circuit that is provided at the distal end portion 11 of the insertion portion 2, and shapes a waveform of a drive signal to the CCD 12.

The waveform shaping circuit 13 is provided with a stabilizing circuit 14 to operate the waveform shaping circuit 13 stably. The stabilizing circuit 14 is a circuit that stabilizes a power supply voltage to the waveform shaping circuit 13. The stabilizing circuit 14 is, for example, a ceramics capacitor. The capacitor which is the stabilizing circuit 14 is connected to between two power supply lines L1 and L2 for power supply of the waveform shaping circuit 13. The power supply line L1 is a line that is connected to a constant voltage source of the main body portion 3, and the power supply line L2 is a line that is connected to a ground (GND).

The horizontal transfer signal φH after shaping is inputted into the CCD 12. An image pickup signal that is outputted from the CCD 12 is outputted to a signal line L3 via a buffer circuit 15. The image pickup signal outputted via the signal line L3 is inputted into an image processing section 24 of the main body portion 3.

Signal lines L0 and L3 and the power supply lines L1 and L2 are connected to the main body portion 3 through an inside of the insertion portion 2 from the distal end portion 11.

The main body portion 3 has a constant voltage source 21. The constant voltage source 21 is a voltage generation circuit for supplying a power supply voltage VD to the waveform shaping circuit 13 via the power supply line L1 which is inserted through the inside of the insertion portion 2.

The main body portion 3 has a constant current source 22 as a dummy load circuit, and a transistor 23 as a switch element. The constant current source 22 configures a load circuit connected to the power supply line L1 which supplies the power supply voltage of the waveform shaping circuit 13. The power supply line L4 is inserted through the inside of the insertion portion 2 together with the signal lines L0 and L3 and the power supply lines L1 and L2, and one end of the power supply line L4 is connected to the power supply line L1 at a connection point P1 in the distal end portion 11. The other end of the power supply line L4 is connected to a drain side of the transistor 23.

A source side of the transistor 23 is connected to the ground via the constant current source 22. The constant current source 22 is a constant current circuit that is configured by a transistor and the like and passes a predetermined current to the power supply line L4.

A gate of the transistor 23 is connected to a timing generator (hereinafter, abbreviated as TG) 26 included in the image processing section 24 of the main body portion 3 by a signal line L5. A switching signal HBLK from the TG 26 is inputted into the gate of the transistor 23. The switching signal HBLK is a load switching signal that switches a load.

The transistor 23 is controlled to be on and off by the switching signal HBLK, and thereby connection of the power supply line L4 which is inserted through the insertion portion 2 and the constant current source 22 is controlled. The transistor 23 and the TG 26 configure a current drawing circuit that draws a predetermined current to the constant current source 22 which is a load circuit from the power supply line L1 during a non-operation time period (T0) of the waveform shaping circuit 13 so that a potential difference between the power supply voltage of the waveform shaping circuit 13 in a clock stop time period T0 that is the non-operation time period of the waveform shaping circuit 13, and the power supply voltage in a clock output time period T1 that is an operation time period of the waveform shaping circuit 13 becomes small, so that the power supply voltage during the non-operation time period of the waveform shaping circuit 13 and the power supply voltage during the operation time period preferably become equal. In other words, the transistor 23 and the TG 26 which configure the current drawing circuit draw the predetermined current to the constant current source 22 from the power supply line L1 in response to an operation state of the waveform shaping circuit 13 so that the potential difference between the power supply voltage of the waveform shaping circuit 13 in the non-operation time period of the waveform shaping circuit 13, and the power supply voltage in the operation time period of the waveform shaping circuit 13 becomes small. Drawing of the predetermined current from the power supply line L1 is performed by drawing the predetermined current to the constant current source 22 which is a load circuit via the power supply line L4 connected to the connection point P1 to the power supply line L1 in the distal end portion 11 of the waveform shaping circuit 13.

Further, the main body portion 3 has the image processing section 24. The image pickup signal from the signal line L3 is inputted into the image processing section 24 via the buffer circuit 25.

The image processing section 24 includes a circuit (not illustrated) for various kinds of image processing, applies various kinds of image processing to the inputted image pickup signal to generate an endoscopic image, and outputs the endoscopic image to a monitor as an image signal. Therefore, the endoscopic image is displayed on the monitor, and an inspector can perform inspection of an object to be inspected by looking at the endoscopic image.

The image processing section 24 has the TG 26 which generates various timing signals in the endoscope apparatus 1. The TG 26 is a circuit that generates various timing signals for use in the endoscope apparatus 1. In FIG. 1, as the timing signals which the TG 26 outputs, only the horizontal transfer signal φH to the signal line L0 and the control signal HBLK to the transistor 23 are shown. The horizontal transfer signal φH from the TG 26 is outputted to the signal line L0 via a buffer circuit 27 that is a drive circuit.

### (Operation)

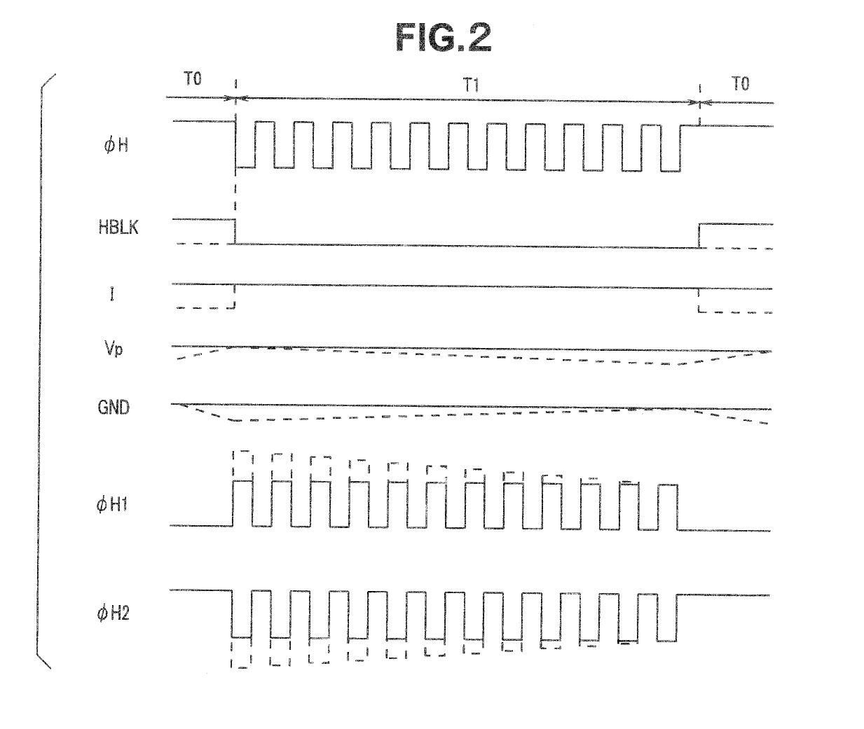

Next, an operation of the endoscope apparatus 1 will be described with use of FIG. 2. FIG. 2 is a waveform chart of various signals in the endoscope apparatus 1. Here, one horizontal transfer signal φH will be described.

The horizontal transfer signal φH which is a shaped signal is outputted from the TG 26. The TG 26 controls output of the horizontal transfer signal φH so that the horizontal transfer signal φH is outputted only in the clock output time period T1 as shown in FIG. 2. In the clock output time period T1, an image pickup signal corresponding to pixels in an effective pixel region of the CCD 12 is outputted from the CCD 12. The time period other than the clock output time period T1 is a clock stop time period T0 in which output of the horizontal transfer signal φH is stopped, and corresponds to a blanking time period of the CCD 12.

Further, the TG 26 is configured so as to output the switching signal HBLK during the clock stop time period T0, and so as not to output the switching signal HBLK during the clock output time period T1.

Therefore, as shown in FIG. 2, the switching signal HBLK becomes high during the clock stop time period T0, and turns on the transistor 23. When the transistor 23 is turned on, the constant current source 22 which is a dummy load circuit draws a current via the power supply line L4 from the power supply line L1 of the distal end portion 11.

Further, the switching signal HBLK becomes low during the clock output time period T1, and the transistor 23 is turned off. However, during the clock output time period T1, the horizontal transfer signal φH is inputted into the waveform shaping circuit 13, and the CCD 12 is driven.

Namely, the power supply voltage VD from the constant voltage source 21 is supplied to the power supply line L1, but during the clock stop time period T0, drawing of a constant current from the power supply line L1 of the waveform shaping circuit 13 is performed by the constant current source 22 which is a dummy load circuit, whereas during the clock output time period T1, drawing of the constant current like this is not performed, but during the clock output time period T1, the waveform shaping circuit 13 consumes a current to drive the CCD 12.

The constant current source 22 which is a dummy load circuit draws a predetermined constant current via the power supply line L4 from the power supply line L1 of the waveform shaping circuit 13 during the clock stop time period T0 so that magnitudes of currents I which flow into the power supply line of the waveform shaping circuit 13 become equal in the clock stop time period T0 and the clock output time period T1. An output current value of the constant current source 22 is set so that the currents I which flow into the power supply line of the waveform shaping circuit 13 become equal in the clock stop time period T0 and the clock output time period T1.

As a result, a potential difference does not occur during the clock output time period T1 and during the clock stop time period T0 to a voltage Vp in the point P of the power supply input terminal of the waveform shaping circuit 13. Therefore, the power supply voltage VD of the waveform shaping circuit 13 is made constant and the waveform shaping circuit 13 can be operated stably.

Conventionally, drawing of the predetermined current from the power supply line L1 of the waveform shaping circuit 13 is not performed during the clock stop time period T0 as described above. Therefore, the phenomenon in which the potential Vp at the point P declines during the clock output time period T1 and rises during the clock stop time period T0 is repeated, and at the same time, the potential of the ground (GND) also repeats rise and decline in the opposite direction from the potential Vp at the point P, as shown by the dotted lines in FIG. 2.

Further, the voltage of the horizontal transfer signal φH also decreases in the clock output time period T1. For example, the respective voltages of the two CCD horizontal transfer signals φH1 and φH2 gradually decline from the start time point of the clock output time period T1 as shown by the dotted lines in the lower stages of FIG. 2.

In contrast with this, according to the aforementioned present embodiment, the predetermined current is drawn to the dummy load circuit from the point to which the power supply voltage VD of the waveform shaping circuit 13 is applied, during the clock stop time period T0. Therefore, the power supply voltage VD of the waveform shaping circuit 13 does not generate a potential difference between the clock output time period T1 and the clock stop time period T0, and can operate the waveform shaping circuit 13 stably.

In particular, at least during a transfer time period of an effective pixel signal, a difference is prevented from occurring to the voltage drop amount of the conductor wire for supplying power supply of the waveform shaping circuit 13, whereby the power supply voltage which is supplied to the waveform shaping circuit 13 can be kept constant, and degradation of image quality of an endoscopic image can be prevented.

Next, modifications of the aforementioned first embodiment will be described.

### (Modification 1)

In the aforementioned first embodiment, the dummy load circuit is provided in the main body portion 3, but the dummy load circuit may be provided in the distal end portion 11.

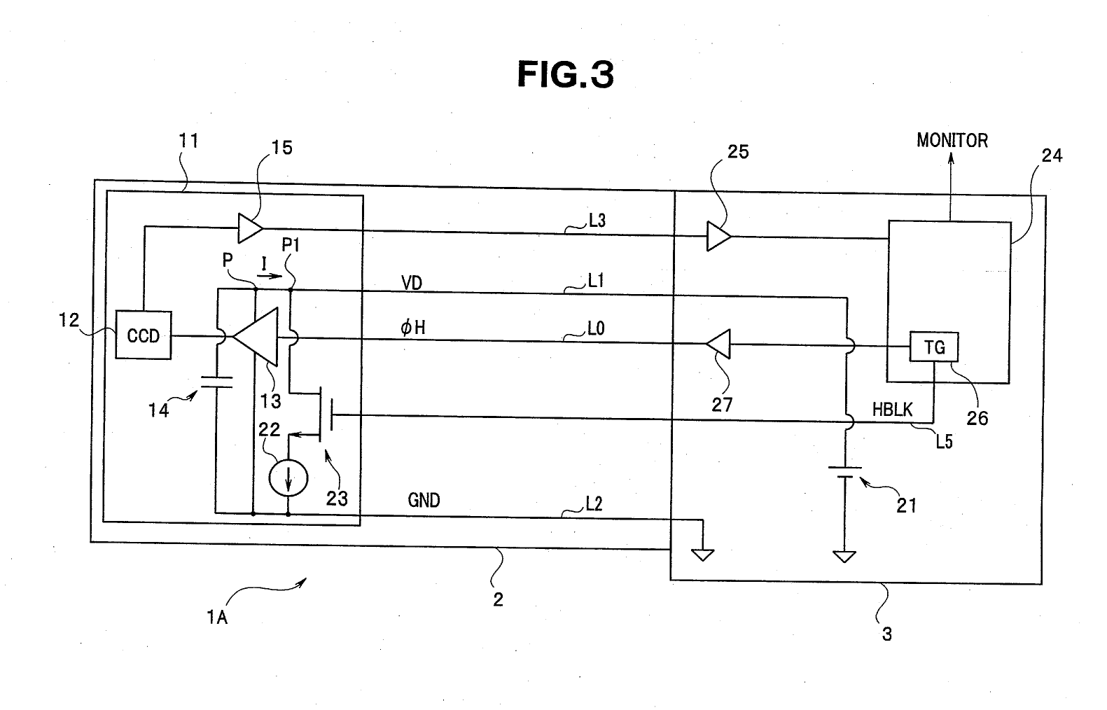

FIG. 3 is a schematic configuration diagram of an endoscope apparatus according to modification 1 of the first embodiment. In FIG. 3, the same components as in FIG. 1 are assigned with the same reference signs, and the description thereof will be omitted. As shown in FIG. 3, in an endoscope apparatus 1A of the modification, the constant current source 22 which is a dummy load circuit, and the transistor 23 are disposed in the distal end portion 11 of the insertion portion 2, the signal line L5 for the switching signal HBLK is inserted through the inside of the insertion portion 2, and one end of the signal line L5 is connected to the TG 26, whereas the other end is connected to the gate of the transistor 23.

In particular, if the waveform shaping circuit 13 and the constant current source 22 are configured by one chip, the advantages that the circuit size of the distal end portion 11 becomes small and the cost can be also reduced are provided.

With the configuration as in FIG. 3, the effect similar to the first embodiment can be also obtained. Further, the ground side is not included in the current loop, and therefore, the effect that the potential of the ground side is stabilized is provided.

### (Modification 2)

An endoscope apparatus 1B of the present modification 2 uses a current that flows into a dummy load circuit, causes a light emitting element such as a light emitting diode (hereinafter, called an LED) to emit light, and uses the light obtained by light emission thereof as a part of illuminating light.

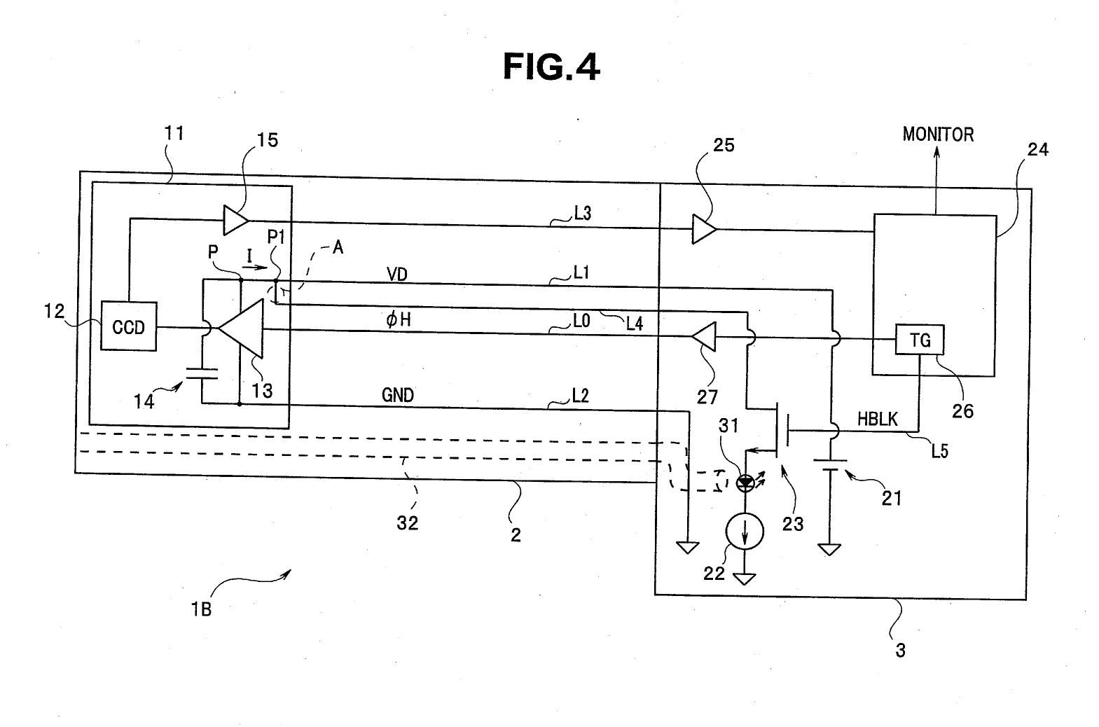

FIG. 4 is a schematic configuration diagram of an endoscope apparatus according to modification 2 of the first embodiment. In FIG. 4, the same components as in FIG. 1 are assigned with the same reference signs and the description will be omitted. As shown in FIG. 4, an LED element 31 is connected in series to the constant current source 22 which is a dummy load circuit. In a vicinity of the LED element 31, one end of a light guide 32 formed from optical fibers is disposed, and the light guide 32 is inserted through the inside of the insertion portion 2. The other end of the light guide 32 is fixed to the distal end of the distal end portion 11. The other end of the light guide 32 is fixed to the distal end portion 11 so that light that is emitted from the other end of the light guide 32 illuminates an image pickup direction of the CCD 12 from the distal end portion 11.

When the transistor 23 is turned on during the clock stop time period T0, a current flows into the LED element 31, and the LED element 31 emits light. The light of the LED element 31 is incident from one end of the light guide 32, and emits from the other end.

Note that the LED element 31 may be provided in a position at a point A shown by the dotted line in FIG. 4. In this case, the effect that the light guide 32 becomes unnecessary is provided.

According to the present modification, the effect similar to the first embodiment can be obtained, and since during the clock stop time period T0, in addition to the illuminating light from the light guide not illustrated, the light emitted from the light guide 32 is added, an object can be irradiated with brighter illuminating light.

### (Modification 3)

An endoscope apparatus IC of the present modification 3 also uses a current that flows into the dummy load circuit, causes an light emitting element such as an LED to emit light, and uses the light obtained by light emission thereof as a part of illuminating light.

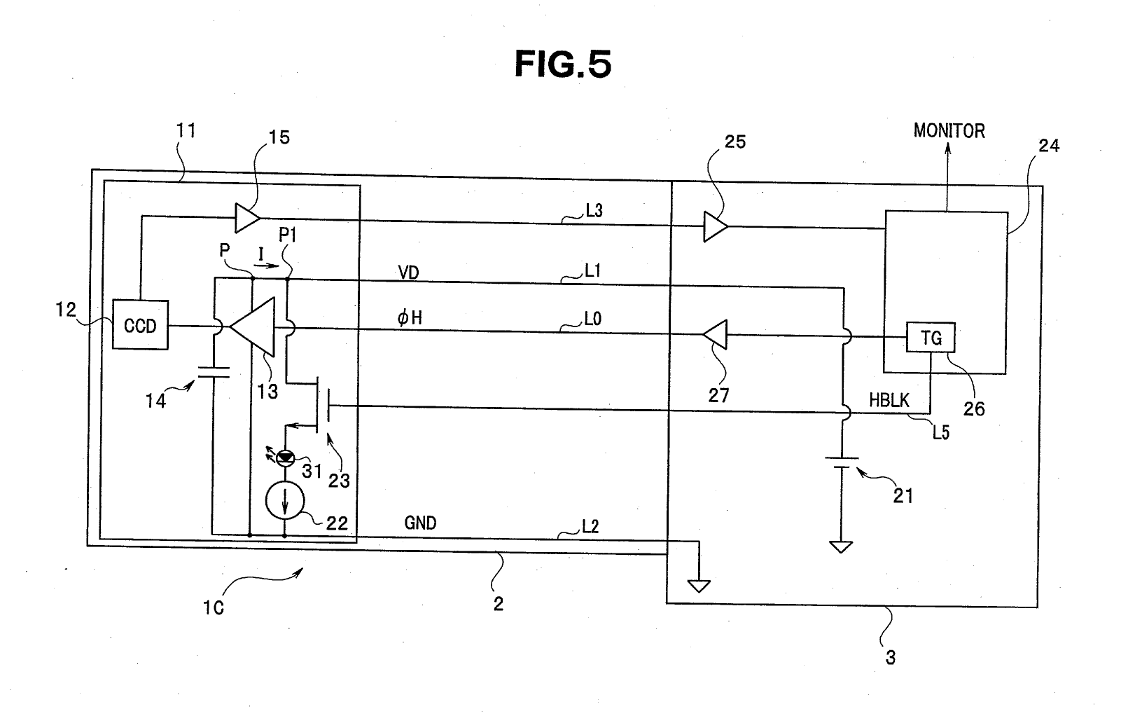

FIG. 5 is a schematic configuration diagram of the endoscope apparatus according to the modification 3 of the first embodiment. FIG. 5 shows a case in which in the aforementioned modification 1, a current that flows into the dummy load circuit is used, a light emitting element such as an LED is caused to emit light, and the light obtained by light emission thereof is used as a part of illuminating light.

The LED 31 is disposed in the distal end portion 11. In particular, the LED 31 is disposed in the distal end portion 11 so that light of the LED 31 is emitted from the distal end of the distal end portion 11. In the LED 31, a current flows into the LED element 31 when the transistor 23 is turned on during the clock stop time period T0, and the LED element 31 emits light. The light of the LED element 31 is emitted from the distal end of the distal end portion 11.

According to the present modification, the effect similar to the first embodiment can be obtained, and since during the clock stop time period T0, in addition to the illuminating light from the light guide not illustrated, the emitted light of the LED element 31 is further added, an object can be irradiated with brighter illuminating light.

### Second Embodiment

While the endoscope apparatus of the first embodiment is configured to draw the predetermined current into the dummy load circuit from the point to which the power supply voltage VD of the waveform shaping circuit 13 is applied during the clock stop time period T0, an endoscope apparatus 1D of a second embodiment is configured to reduce a power supply voltage by a predetermined amount during the clock stop time period T0.

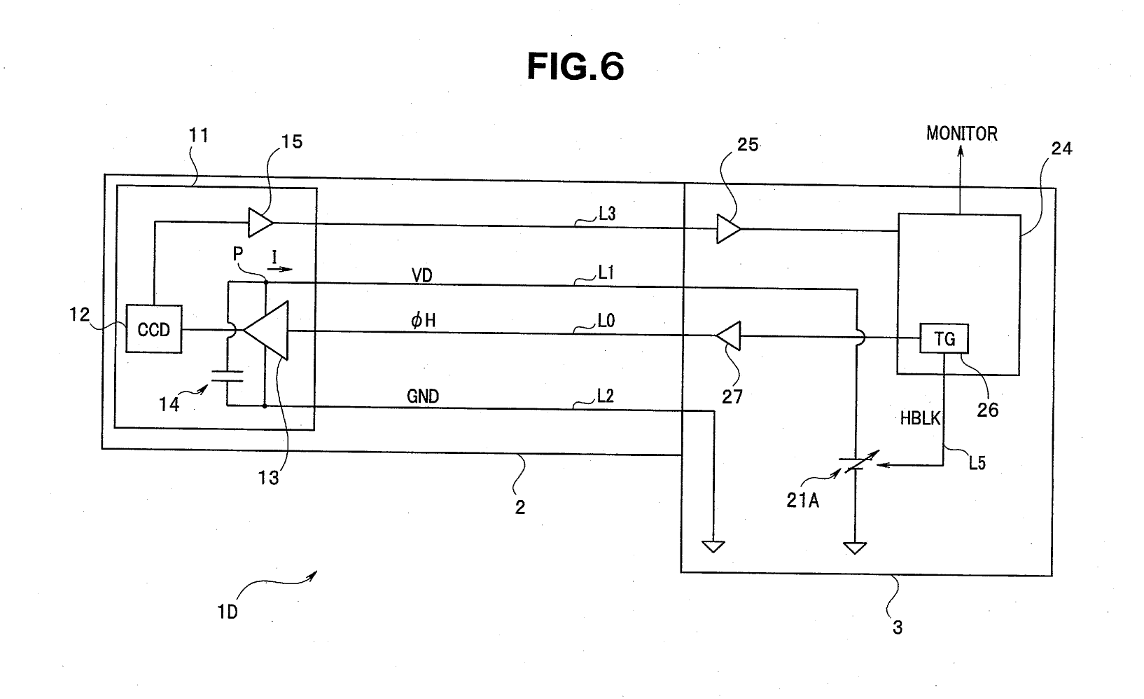

FIG. 6 is a schematic configuration diagram of an endoscope apparatus according to the second embodiment of the present invention. In FIG. 6, the same components as in FIG. 1 are assigned with the same reference signs, and the description will be omitted. As shown in FIG. 6, a constant voltage source 21A of the main body portion 3 of the endoscope apparatus 1D is a constant voltage circuit that can change an output voltage in two steps.

The constant voltage source 21A is a constant voltage circuit that changes an output voltage Vs in response to the switching signal HBLK. When the switching signal HBLK is low, the constant voltage source 21A outputs a predetermined constant voltage V1, and when the switching signal HBLK is high, the constant voltage source 21A outputs a constant voltage V2 which is lower than the predetermined constant voltage V1 by a predetermined voltage dV.

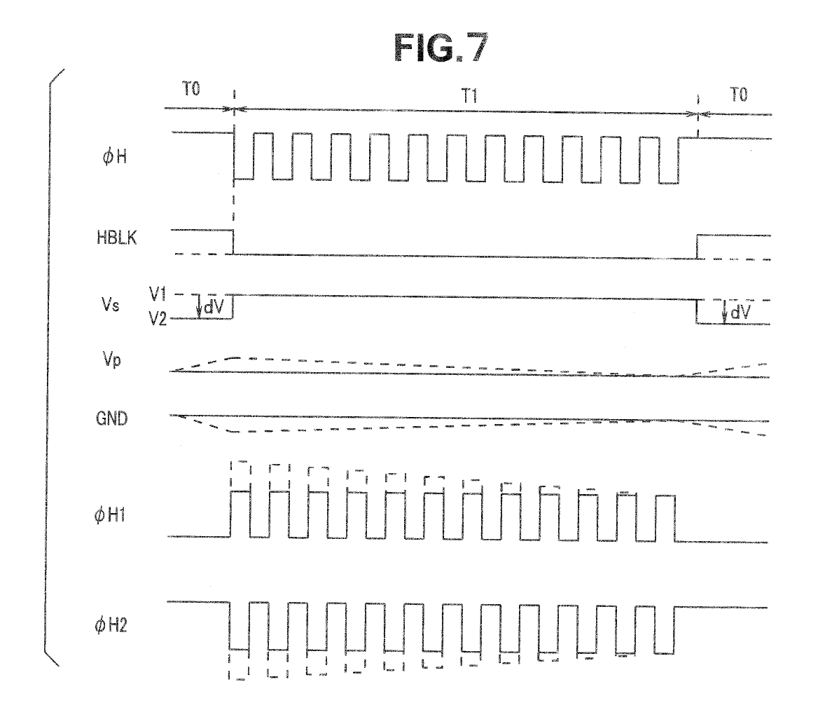

An operation of the above described endoscope apparatus 1D will be described with use of FIG. 7. FIG. 7 is a waveform chart of various signals in the endoscope apparatus 1D.

When the switching signal HBLK from the TG 26 is high, the constant voltage source 21A outputs a constant voltage of the voltage V2, and when the switching signal HBLK from the TG 26 is low, the constant voltage source 21A outputs a constant voltage higher than the voltage V2 by the predetermined voltage dV.

Namely, in the clock stop time period T0, the power supply voltage of the waveform shaping circuit 13 is made low, and an electric charge amount stored in the stabilizing circuit 14 is decreased. Therefore, during the clock stop time period T0, rise of the voltage Vp at the point P can be suppressed.

As a result, the endoscope apparatus which can keep the power supply voltage, which is supplied to the waveform shaping circuit, constant can be realized.

Namely, the TG 26 and the constant voltage source 21A configure a variable voltage source that reduces the power supply voltage which is supplied to the waveform shaping circuit 13 during the non-operation time period (T0) of the waveform shaping circuit 13 so that the potential difference between the power supply voltage of the waveform shaping circuit 13 in the clock stop time period T0 which is the non-operation time period of the waveform shaping circuit 13, and the power supply voltage of the waveform shaping circuit 13 in the clock output time period T1 which is the operation time period of the waveform shaping circuit 13 becomes small, and preferably so that the power supply voltage during the non-operation time period of the waveform shaping circuit 13 and the power supply voltage during the operation time period become equal. In other words, the TG 26 and the constant voltage source 21A which configure the variable voltage source changes the power supply voltage in response to the operation state of the waveform shaping circuit 13 so that the potential difference between the power supply voltage of the waveform shaping circuit 13 in the non-operation time period of the waveform shaping circuit 13, and the power supply voltage of the waveform shaping circuit 13 in the operation time period of the waveform shaping circuit 13 becomes small.

Consequently, according to the present embodiment described above, the power supply voltage which is supplied to the waveform shaping circuit 13 is reduced during the clock stop time period T0. Therefore, a potential difference does not occur to the power supply voltage VD of the waveform shaping circuit 13 between the clock output time period T1 and the clock stop time period T0, and the waveform shaping circuit 13 can be stably operated.

In particular, at least during the transfer time period of an effective pixel signal, the power supply voltage which is supplied to the waveform shaping circuit 13 can be kept constant, and degradation of the image quality of an endoscopic image can be prevented.

In particular, at least during the transfer time period of the effective pixel signal, a difference is prevented from occurring to the voltage drop amount of the conductor wire for supplying power supply of the waveform shaping circuit 13, whereby the power supply voltage which is supplied to the waveform shaping circuit 13 can be kept constant, and degradation of the image quality of an endoscopic image can be prevented.

Further, in the endoscope apparatus 1D of the present embodiment, the signal line L4 or L5 is not present in the inside of the insertion portion 2, as compared with the first embodiment, and therefore, reduction in the diameter of the insertion portion 2 can be achieved.

### (Modification 1)

An endoscope apparatus 1E of the present modification 1 uses a current that flows into the constant voltage source 21A, causes a light emitting element such as an LED to emit light, and uses the light obtained by light emission thereof as a part of illuminating light.

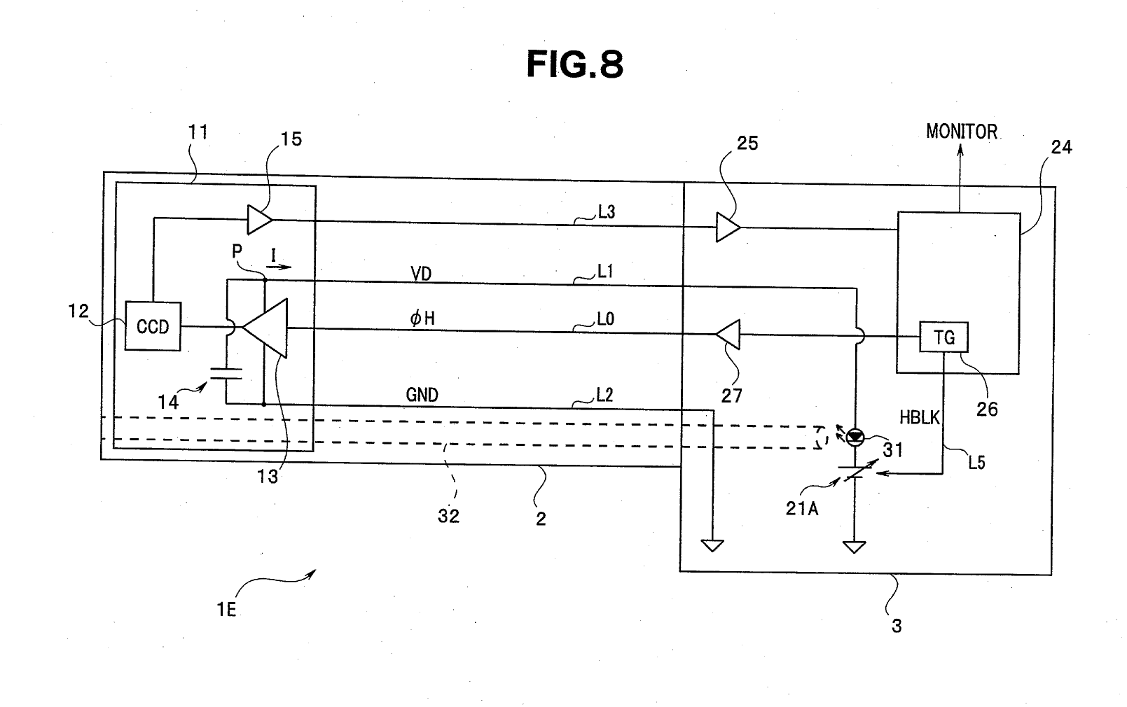

FIG. 8 is a schematic configuration diagram of the endoscope apparatus 1E according to modification 1 of the second embodiment. In FIG. 8, the same components as in FIG. 6 are assigned with the same reference signs, and the description will be omitted. As shown in FIG. 8, the LED element 31 is connected in series to the constant voltage source 21A. In the vicinity of the LED element 31, one end of the light guide 32 formed from optical fibers is disposed, and the light guide 32 is inserted through the inside of the insertion portion 2. The other end of the light guide 32 is fixed to the distal end of the distal end portion 11. The other end of the light guide 32 is fixed to the distal end portion 11 so that light emitted from the other end of the light guide 32 illuminates the image pickup direction of the CCD 12 from the distal end portion 11.

When the transistor 23 is turned on during the clock stop time period T0, a current flows into the LED element 31, and the LED element 31 emits light. The light of the LED element 31 is incident from one end of the light guide 32, and emits from the other end.

Consequently, according to the present modification, the effect similar to the second embodiment can be obtained, and since during the clock stop time period T0, in addition to the illuminating light from the light guide not illustrated, the light emitted from the light guide 32 is also added, an object can be irradiated with brighter illuminating light.

### (Modification 2)

An endoscope apparatus 1F of the present modification 2 also uses a current that flows into the constant voltage source 21, causes an light emitting element such as an LED to emit light, and uses the light obtained by light emission thereof as a part of illuminating light.

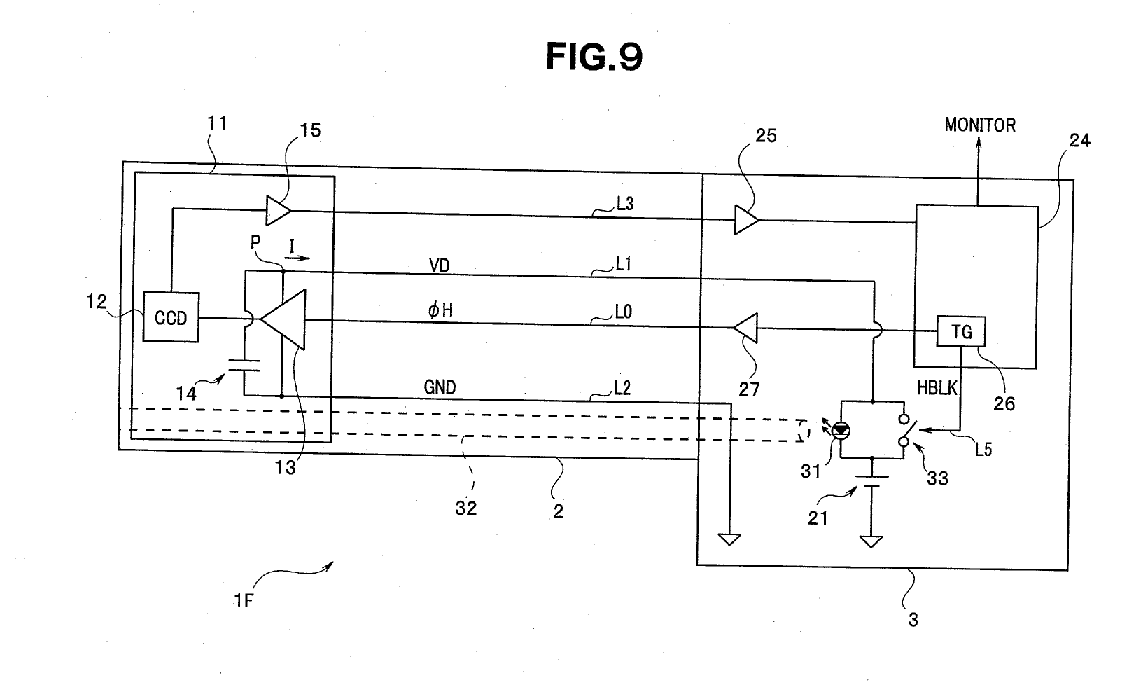

FIG. 9 is a schematic configuration diagram of an endoscope apparatus according to modification 2 of the second embodiment. FIG. 9 is a diagram showing a case in which in the second embodiment described above, the current flowing into the constant voltage source 21 is used, the light emitting element such as an LED is caused to emit light, and the light obtained by light emission thereof is used as a part of illuminating light.

In order to make a constant voltage which is supplied to the waveform shaping circuit 13 differ in the clock output time period T1 and the clock stop time period T0, voltage drop of a forward voltage Vf that is a voltage between an anode and a cathode in the LED element 31 is used.

Therefore, a parallel circuit of the LED element 31 and a switch 33 is connected in series to the constant voltage source 21, and in order that the switch 33 is on during the clock output time period T1, the switching signal HBLK from the TG 26 is supplied to the switch 33. Namely, a variable voltage source is configured by the LED element 31, the switch 33, the constant voltage source 21 and the TG 26.

In the vicinity of the LED element 31, one end of the light guide 32 is disposed, and the light guide 32 is inserted through the inside of the insertion portion 2. The other end of the light guide 32 is fixed to the distal end of the distal end portion 11. The other end of the light guide 32 is fixed to the distal end portion 11 so that light that is emitted from the other end of the light guide 32 illuminates the image pickup direction of the CCD 12 from the distal end portion 11.

During the clock output time period T1, the switch 33 is on, and the voltage V1 is supplied to the waveform shaping circuit 13.

When the switch 33 is turned off during the clock stop time period T0, a current flows into the LED element 31, the LED element 31 emits light, and the voltage V2 which is lower than the voltage V1 is supplied to the waveform shaping circuit 13. The light of the LED element 31 is incident from one end of the light guide 32, and emits from the other end. Namely, the variable voltage source includes the constant voltage source 21 and the LED element 31 which is a light emitting element, and connection of the constant voltage source 21 and the LED element 31 is controlled, whereby the power supply voltage which is supplied to the waveform shaping circuit 13 is reduced.

The number of LED elements 31 is set so that currents I that flow into the power supply line of the waveform shaping circuit 13 become equal in the clock stop time period T0 and the clock output time period T1.

Consequently, according to the present modification, the effect similar to the second embodiment can be obtained, and since in the clock stop time period T0, in addition to the illuminating light from the light guide not illustrated, the light which is emitted from the light guide 32 is added, an object can be irradiated with brighter illuminating light.

As above, according to the plurality of embodiments and the respective modifications of the respective embodiments described above, the endoscope apparatus which can keep the power supply voltage which is supplied to the waveform shaping circuit constant by preventing a difference from occurring to the voltage drop amount of the conductor wire for supplying power supply can be provided.

In particular, even if the number of pixels of an image pickup device is increased, the power supply voltage of the waveform shaping circuit is stabilized, and degradation of the image quality of the endoscopic image can be prevented.

The present invention is not limited to the embodiments described above, and various modifications, alterations and the like can be made within the range without departing from the gist of the present invention.

What is claimed is:

1. An endoscope apparatus having an insertion portion having an image pickup device and a circuit in a distal end portion, and a main body portion, comprising:
a stabilizing circuit that stabilizes a power supply voltage to the circuit;
a load circuit that is connected to a power supply line that supplies the power supply voltage of the circuit; and
a current drawing circuit that draws a predetermined current to the load circuit from the power supply line in response to an operation state of the circuit so that a potential difference between a power supply voltage in a non-operation time period of the circuit and a power supply voltage in an operation time period of the circuit becomes small.

2. The endoscope apparatus according to claim 1,
wherein the circuit is a waveform shaping circuit that shapes a waveform of a drive signal to the image pickup device.

3. The endoscope apparatus according to claim 1,
wherein the current drawing circuit draws the predetermined current to the load circuit so that the power supply voltage in the non-operation time period of the circuit and the power supply voltage in the operation time period of the circuit become equal.

4. The endoscope apparatus according to claim 1,
wherein the load circuit is provided in the main body portion.

5. The endoscope apparatus according to claim 2,
wherein the load circuit is provided in the main body portion, and
the current drawing circuit performs drawing of the predetermined current from the power supply line by drawing the predetermined current to the load circuit via a line connected to a connection point to the power supply line in the distal end portion of the waveform shaping circuit.

6. The endoscope apparatus according to claim 1,
wherein the load circuit is provided in the distal end portion.

7. The endoscope apparatus according to claim 1,
wherein the load circuit has a light emitting element.

8. The endoscope apparatus according to claim 7,
wherein the light emitting element is provided in the main body portion, and
the endoscope apparatus further comprises a light guide for emitting emission light of the light emitting element from the distal end portion.

9. The endoscope apparatus according to claim 7,
wherein the light emitting element is provided in the distal end portion.

10. The endoscope apparatus according to claim 7,
wherein the load circuit and the light emitting element are provided in the distal end portion.

11. An endoscope apparatus having an insertion portion having an image pickup device in a distal end portion, and a main body portion, comprising:
a variable voltage source in the main body portion,
wherein the variable voltage source changes a power supply voltage in response to an operation state of the distal end portion so that a potential difference of power supply voltages before and after change of an operation of the distal end portion becomes small.

12. The endoscope apparatus according to claim 11,
wherein the variable voltage source changes the power supply voltage in response to the operation state of the distal end portion so that the power supply voltages become equal before and after the change of the operation of the distal end portion.

13. The endoscope apparatus according to claim 12, further comprising:
a circuit that is provided in the distal end portion; and
a stabilizing circuit that stabilizes a power supply voltage to the circuit,
wherein the variable voltage source changes the power supply voltage to the circuit in response to an operation state of the circuit.

14. The endoscope apparatus according to claim 13,
wherein the circuit is a waveform shaping circuit that shapes a waveform of a drive signal to the image pickup device.

15. The endoscope apparatus according to claim 13,
wherein the variable voltage source reduces the power supply voltage which is supplied to the circuit during a non-operation time period of the circuit so that the power supply voltage in the non-operation time period of the circuit and the power supply voltage in an operation time period of the circuit become equal.

16. The endoscope apparatus according to claim 11, further comprising:
a light emitting element that is connected to the variable voltage source.

17. The endoscope apparatus according to claim 16,
wherein the light emitting element is provided in the main body portion, and
the endoscope apparatus further comprises a light guide for emitting emission light of the light emitting element from the distal end portion.

18. The endoscope apparatus according to claim 14,
wherein the variable voltage source includes a constant voltage source and a light emitting element, and reduces the power supply voltage which is supplied to the waveform shaping circuit by controlling connection of the constant voltage source and the light emitting element.
